(19) United States
(12) Patent Application Publication
(10) Pub. No.: US 2010/0042161 A1
(43) Pub. Date: Feb. 18, 2010

(54) SURGICAL APPARATUS FOR OSTEOSYNTHESIS

(76) Inventor: Alexandre Worcel, Montlignon (FR)

Correspondence Address: YOUNG & THOMPSON, 209 Madison Street, Suite 500, Alexandria, VA, 22314, US

(21) Appl. No.: 12/440,382

(22) PCT Filed: Sep. 7, 2007
(86) PCT No.: PCT/FR07/01450

§ 371 Date: Jul. 27, 2009

(30) Foreign Application Priority Data
Sep. 8, 2006 (FR) 0607873

(51) Int. Cl. A61B 17/80

(52) U.S. Cl. 606/289

(57) ABSTRACT

A surgical apparatus for osteosynthesis, includes a plate (2) provided with at least two holes and intended to join two bone parts, and a blocking sleeve (10) which is substantially of revolution and is intended to be screwed into one of the holes of the plate, the blocking sleeve having an axial orifice for passage of a pin to be screwed into one of the bone parts, wherein the lower part of the blocking sleeve has a slit along at least one of its radii, and the blocking sleeve and the associated hole cooperate by threaded engagement in order to block the sleeve in the hole when the sleeve is screwed into the hole.

The present invention relates to a surgical apparatus for osteosynthesis, comprising a plate and a blocking sleeve. It also relates to an apparatus further comprising a pin, the blocking sleeve being intended to ensure blocking between the plate and the pin.

Various devices from the prior art are known which are able to provide such an attachment.

Thus, the use of conventional type screws has been proposed, the head of which is milled at 45° so that it may be embedded inside a recess of the same shape provided in the plate. However it is found that the axis of the threaded hole made in the bone portion does not strictly coincide with that of the frustro-conical cavity made in the plate, stresses are generated at the bone portion, the latter being capable over time of undermining the integrity of the bone portion and consequently being detrimental to proper attachment of the plate.

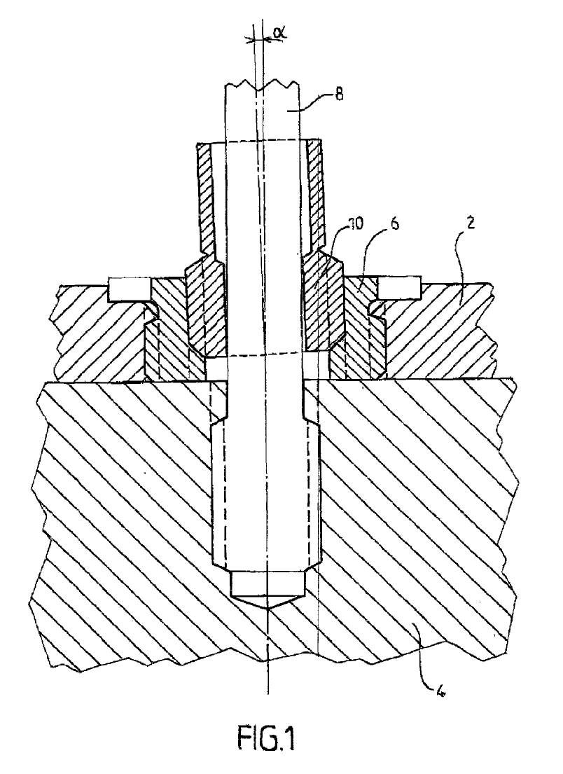

An angular shift of the axes of the pin and of the sleeve was proposed in order to find a remedy to these drawbacks. Such a device is disclosed in document EP-1 583 478 and is illustrated in FIG. 1. This osteosynthesis device comprises a plate 2 including at least two holes, only one of which is illustrated in the figure, intended to receive pins which are screwed into a bone portion.

The holes of the plate area are threaded and receive an intermediate sleeve 6. The latter has a central orifice which is itself threaded, the zz′ axis of which is tilted by an angle α relatively to its axis of revolution yy′.

Finally, a blocking sleeve 10 is screwed by threaded engagement to its lower portion in the intermediate sleeve.

In order to place the osteosynthesis device, the practitioner after having perforated the plate 2 in at least two locations selected for providing its attachment on the bone portion 4, performs drilling of this bone portion by guiding a drill perpendicularly to the plate. Next, he/she places the intermediate sleeve 6 and screws the threaded pin 8 into the bone portion up to the desired length. He/she then introduces the blocking sleeve 10 on the pin 8 and proceeds with screwing this blocking sleeve 10 into the intermediate sleeve 6.

It is understood that because of the angular shift α existing between the respective axes yy′ and zz′, gradual jamming of the blocking sleeve 10 is achieved during this screwing in the intermediate sleeve 6. Irreversible blocking of the pin 8 is thereby achieved relatively to the plate 2.

This device is efficient but it has a certain number of drawbacks.

First of all, tolerance is low on the angular gap between the axes yy′ and zz′. Manufacturing the intermediate sleeve is therefore delicate, which results in high manufacturing cost.

Moreover, this angular gap while ensuring efficient blocking has the drawback that this blocking already occurs right at the beginning of the screwing of the blocking sleeve 10. Therefore, only insufficient penetration of the blocking sleeve into the plate 2 may occur.

Finally, in the case of a plate with a small thickness (for example less than 3 mm), the intermediate sleeve and the blocking sleeve have to be partly inserted into the bone portion so as to allow the device to be properly locked. With this, it is not possible to use this prior art device for the upper bones (arms, forearms, hands) which are too thin.

The object of the invention is to do away with the limitations of the osteosynthesis devices of the prior art.

For this purpose, the object of the invention is a surgical apparatus for osteosynthesis comprising a plate provided with at least two holes intended to join two bone portions and with at least one blocking substantially axisymmetrical sleeve, intended to be screwed into one of the holes of the plate, said blocking sleeve including an axial orifice intended for letting through a pin to be screwed into one of the bone portions, wherein:
a lower portion of the blocking sleeve includes a slit along at least one of its radii, and
said blocking sleeve and the associated hole cooperate by threaded engagement in order to block the blocking sleeve in the hole when the sleeve is screwed into the hole.

Advantageously, the slit of said blocking sleeve is diametrical.

According to a preferred embodiment, said blocking sleeve has in a lower portion a smaller casing than in an upper portion. In particular, said blocking sleeve has a conical or a semi-hemispherical casing.

Alternatively, according to another preferred embodiment, said blocking sleeve has a cylindrical casing and the hole, a beveled casing.

Advantageously, said blocking sleeve and the edges of the holes of the plate have different hardness. In particular, one of the parts among said blocking sleeve and an edge of the plate intended to receive the sleeve is treated in order to increase its hardness, notably by treating its surface with carbon or by work hardening.

According to a particular embodiment, an intermediate sleeve is screwed into the hole of the plate and is intended to cooperate by threaded engagement with the blocking sleeve.

Advantageously, the apparatus according to the invention further includes a pin intended to be screwed into a bone portion.

Preferably, the inner casing of the blocking sleeve and the outer casing of the pin are selected so that the sleeve initially engages with the pin without any friction.

The invention will be better understood upon reading the following description of embodiments of the invention, described as non-limiting examples, with reference to the appended drawings, wherein:

FIG. 1, already described, illustrates a surgical apparatus for osteosynthesis according to the prior art,

FIG. 2 illustrates a surgical apparatus for osteosynthesis according to the invention,

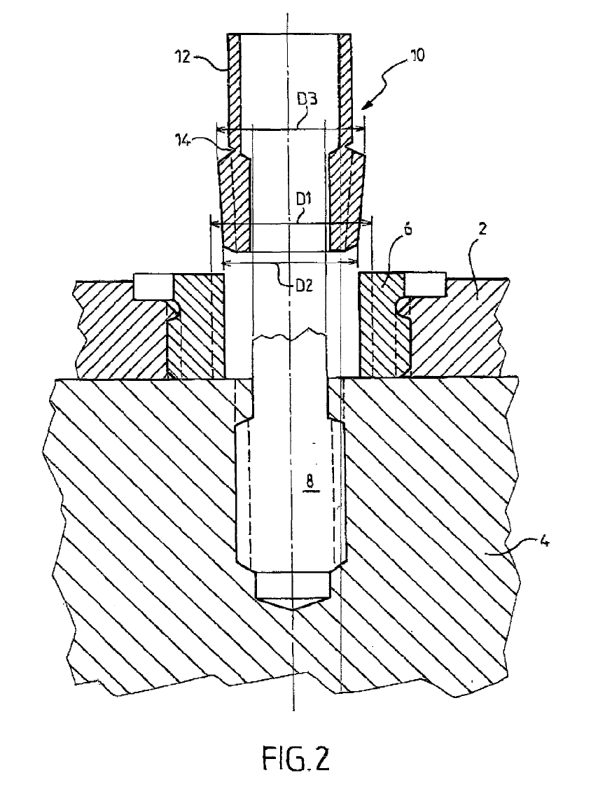

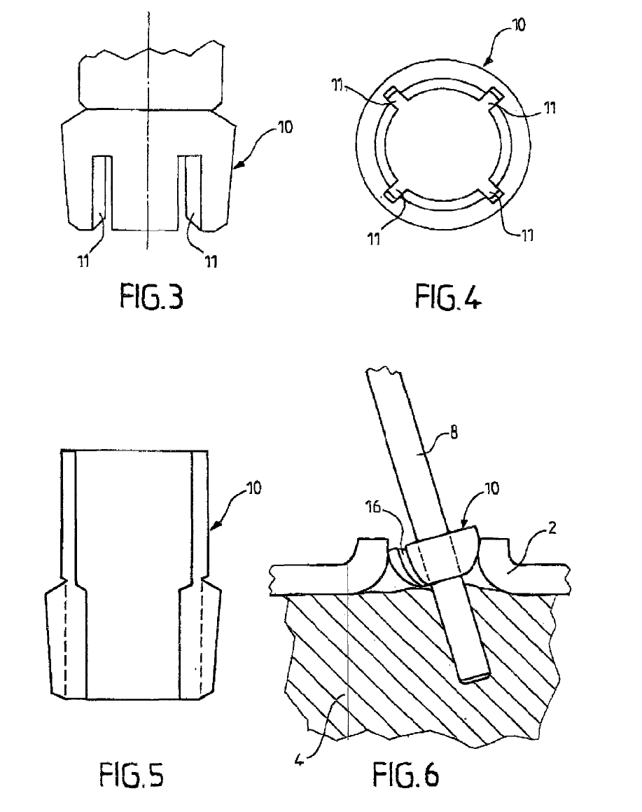

FIGS. 3 and 4 respectively illustrate a side view and a bottom view of the blocking sleeve of the apparatus of FIG. 2,

FIG. 5 illustrates an alternative embodiment of the blocking sleeve according to the invention, and

FIG. 6 illustrates another embodiment of a surgical apparatus for osteosynthesis according to the invention.

FIG. 2 illustrates a sectional view of a surgical apparatus for osteosynthesis according to the invention. It comprises a plate 2 provided with two holes (only one of which is illustrated) each intended to receive a pin 8 to be screwed into the bone portion 4.

In each hole, the practitioner will introduce a blocking sleeve 10 in order to maintain the plate and the pin together. As illustrated, the apparatus may also comprise an intermediate sleeve 6. The latter, although not being essential to the invention, is advantageous in that it provides better dimensional control of the hole receiving the blocking sleeve, than if the blocking sleeve 10 were be directly screwed into the hole made by the practitioner in the plate 2.

According to the invention, the blocking sleeve comprises at least one slit in its lower portion. As an example, the blocking sleeve may comprise 4 slits as illustrated in the side view of FIG. 3 (only two slits are visible) or in the bottom view of FIG. 4.

Moreover, the intermediate sleeve 6 and the blocking sleeve 10 cooperate by threaded engagement when the blocking sleeve is screwed into the hole defined by the intermediate sleeve.

From a practical point of view, it is possible to proceed in the following way: (a) the blocking sleeve 10 is placed by partly screwing it on the intermediate sleeve 6, the screwing-in being sufficiently slight in order not to interfere with the passage of the pin into the axial orifice of the blocking sleeve 10, (b) the pin 8 is screwed into the bone portion, and (c) the screwing-in of the blocking sleeve 10 is completed. It would also be possible to first screw the pin 8, and then from the top of the pin 8 have the blocking sleeve 10 slide along this pin and screw the blocking sleeve 10 on the intermediate sleeve 6.

In order to facilitate this screwing, the blocking sleeve 10 is provided in its upper portion with a gripping means 12 capable of being set into rotation by means of a screwdriver. This upper portion forms a tool but is not part of the surgical apparatus for osteosynthesis after it has been placed. After the sleeve has been screwed in, its upper portion is removed by shearing at a circular area with less resistance 14.

In the embodiment illustrated in FIG. 2, each of the two sleeves includes a threading. It is understood that this is not mandatory. It is sufficient that one of the sleeves has a thread and that the other sleeve is in a softer material so that the thread meshes into this other sleeve.

In the technical field of the invention, it is for example possible to use non-work-hardened 316L steel which has a tensile strength of the order of 45 kg/mm², for one of the sleeves and work-hardened 316L steel, which has a tensile strength of the order of 145 kg/mm² for the other sleeve. The same steel may also be used for both sleeves and one of them may be hardened at least in its portion intended to cooperate with the other sleeve, by a carbon treatment.

Advantageously, the blocking sleeve 10 has starting from its lower face (the one facing the bone portion) and upwards an external casing which increases in size. In other words, the diameter D2 of the blocking sleeve in its lower portion is smaller than the diameter D3 in its upper portion. For example, the casing of the sleeve may be conical or hemispherical.

Thus, the blocking sleeve 10 may be engaged by sliding on the pin 8 without any friction or with low friction, into the hole of the plate and it adheres more and more firmly to the intermediate sleeve 6 as it is gradually screwed in. The radial stresses then exerted on the blocking sleeve tend to close the slits of the latter which reduces its diameter at the slits and effectively locks the blocking sleeve on the pin 8.

The variable casing of the blocking sleeve 10 may either be obtained by using a sleeve with a variable diameter and a constant thread, as illustrated in FIG. 2, or by using a sleeve with a constant diameter but with a gradual thread, i.e. the threading of which is radially larger in the upper portion of the sleeve than in the lower portion of the latter, as illustrated in FIG. 5.

Although a blocking sleeve with a variable casing has been described, it is clear that the casing of the blocking sleeve may be constant and that it is the inner casing of the intermediate sleeve which is variable or the casing of the hole made in the plate when the apparatus is without any intermediate sleeve, the latter as indicated above not being an essential component of the apparatus of the invention.

In FIG. 6 another embodiment of an apparatus according to the invention is illustrated, comprising a plate 2, a pin 8 screwed into a bone portion 4, and a blocking sleeve 10 including a slit 16 which, in the illustrated example, extends over the whole height of this sleeve. The hemispherical shape of the blocking sleeve is particularly of interest in that it allows effective blocking even when the pin is not perpendicular to the plane of the plate.

1. A surgical apparatus for osteosynthesis comprising a plate (2) provided with at least two holes intended to join two bone portions and at least one blocking substantially axisymmetrical sleeve (10), intended to be screwed into one of the holes of the plate, said blocking sleeve including an axial orifice intended to allow the passage of a pin to be screwed into one of the bone portions,
characterized in that, for at least one blocking sleeve:
a lower portion of the blocking sleeve includes a slit (11, 16) along at least one of its radii, and
said blocking sleeve and the associated hole cooperate by threaded engagement in order to block the blocking sleeve in the hole when the sleeve is screwed into the hole.

2. The apparatus according to claim 1, characterized in that the slit of said blocking sleeve is diametrical.

3. The apparatus according to claim 1, characterized in that said blocking sleeve has in a lower portion a smaller casing than in an upper portion.

4. The apparatus according to claim 1, characterized in that said blocking sleeve has a conical casing.

5. The apparatus according to claim 1, characterized in that said blocking sleeve has a semi-hemispherical casing.

6. The apparatus according to claim 1, characterized in that said blocking sleeve has a cylindrical casing and the hole has a beveled casing.

7. The apparatus according to claim 1, characterized in that said blocking sleeve and the edges of the holes of the plate have a different hardness.

8. The apparatus according to claim 7, characterized in that one of the parts among said blocking sleeve and an edge of the hole of the plate intended to receive the sleeve is treated in order to increase its hardness.

9. The apparatus according to claim 8, characterized in that said part is hardened by treatment of its surface with carbon.

10. The apparatus according to claim 8, characterized in that said part is hardened by work-hardening.

11. The apparatus according to claim 1, characterized in that an intermediate sleeve (6) is screwed into the hole of the plate and is intended to cooperate by threaded engagement with the blocking sleeve.

12. The apparatus according to claim 1, characterized in that said apparatus further includes a pin (8) intended to be screwed into the bone portion.

13. The apparatus according to claim 12, characterized in that the inner casing of the blocking sleeve and the outer casing of the pin are selected so that the sleeve initially engages with the pin without any friction.

14. The apparatus according to claim 2, characterized in that said blocking sleeve has in a lower portion a smaller casing than in an upper portion.
